(19) United States
(12) Patent Application Publication
(10) Pub. No.: US 2013/0145511 A1
(43) Pub. Date: Jun. 13, 2013

(54) Eye Protection Device for Sports Helmet

(71) Applicant: John Ross Guenther, Saskatoon (CA)

(72) Inventor: John Ross Guenther, Saskatoon (CA)

(21) Appl. No.: 13/686,306

(22) Filed: Nov. 27, 2012

(30) Foreign Application Priority Data
Nov. 28, 2011 (CA) 2759735

(51) Int. Cl. A61F 9/02

(52) U.S. Cl. CPC A61F 9/029
USPC 2/10; 2/442

(57) ABSTRACT

The present invention seeks to provide an eye protection device that can be removably attached to a standard sports helmet. The device is provided with attachment arms that are configured for connection to existing attachment points on standard helmets.

### FIELD OF THE INVENTION

The present invention relates to eye protection devices, and specifically eye protection devices that are integrated into sports headgear.

### BACKGROUND OF THE INVENTION

It is well known in the field of sports headgear design to provide impact-resistant visors that help to protect a player from injuries to the face and specifically the eyes, such as in the sport of ice hockey. Visors in common use have the advantage of not unduly restricting a player's vision, and they have accordingly gained wide popularity. However, visors are also known for leaving a large gap between the inner surface of the visor and the player's face, with the risk that objects such as hockey sticks and pucks can still be introduced to the face and cause severe injury. In addition, standard visors are bulky and add weight to a helmet and are therefore less desirable than other forms of eye protection. To address this, some players have attempted to use commonly available sports safety glasses, which provide a closer fit to the player's face and hence counter this risk, but it has been found that such glasses are uncomfortable to wear due to the presence of the helmet and cannot be attached securely to the helmet.

It is clear that there is a need for an improved eye protection solution that is both comfortable to wear and effective at reducing injury. Various eye protection devices are known outside the field of sports headgear. For example, Canadian Patent Application No. 2,668,435 to Stachler et al. teaches eye shields that are mounted on the underside of a helmet brim, for pivotable movement between stored and in-use positions; however, adapting this to a sports helmet would not address the risks inherent in standard visor use. Canadian Patent Application No. 2,747,869 to Jenkins et al. teaches mounting means for attaching standard glasses to a safety helmet, but this would not be readily adaptable to a sports helmet given the fact that the latter extends over or around a user's ears whereas a safety helmet rests above the ears.

What is needed, therefore, is a device that can provide protection to a player's eyes, in a form that is comfortable and simple to attach and detach from a helmet.

### SUMMARY OF THE INVENTION

The present invention therefore seeks to provide an eye protection device that is configured to attach to standard helmets and is comfortable for the user.

According to a first aspect of the present invention there is provided an eye protection device for removable attachment to a sports helmet, the device comprising:

a frame;

a transparent viewing lens held within the frame; and

first and second attachment arms having respective anterior and posterior ends;

the attachment arms connected at their respective anterior ends to the frame at laterally spaced connection locations on the frame; and

the attachment arms comprising attachment means at their respective posterior ends for removable attachment to the sports helmet.

In exemplary embodiments of the present invention, the attachment arms have a length sufficient to reach the lateral sides of the helmet to enable attachment. The attachment means preferably comprise a hole or slot in the posterior end of the attachment arm to receive a bolt of other mounting mechanism found on a standard helmet, but they make take other forms such as clips. The attachment arms are preferably of a thick vertical width to assist in protecting the side of the user's face. The viewing lens is preferably made of an impact-resistant material such as polycarbonate, with an anti-scratch and anti-fog coating. The frame preferably includes a nose bridge support for comfort and a rounded bottom edge to help avoid injury to a player's face in impact situations.

A detailed description of exemplary embodiments of the present invention are given in the following. It is to be understood, however, that the invention is not to be construed as being limited to these embodiments.

### BRIEF DESCRIPTION OF THE DRAWINGS

In the accompanying drawings, which illustrate exemplary embodiments of the present invention:

FIG. 1a is a front elevation view of an eye protection device in accordance with the present invention;

FIG. 1b is a photographic perspective view of the eye protection device of FIG. 1a;

FIG. 2 is a side elevation view of the eye protection device of FIG. 1a;

FIG. 3 is a top plan view of the eye protection device of FIG. 1a;

FIG. 4 is a detailed side elevation view of an attachment arm in accordance with the present invention;

FIG. 5 is a side elevation view of a sports helmet being provided with an eye protection device in accordance with the present invention;

FIG. 6 is a detailed side elevation view of a first alternative attachment arm in accordance with the present invention; and

FIG. 7 is a detailed side elevation view of a second alternative attachment arm in accordance with the present invention.

Preferred embodiments of the present invention will now be described with reference to the accompanying drawings.

### DETAILED DESCRIPTION OF EXEMPLARY EMBODIMENT

Referring now to the accompanying drawings, an embodiment of an eye protection device according to the present invention is illustrated. Specific distance and angle measurements are included in some of the Figures, but these are presented simply to further define the exemplary embodiment and are not to be construed as limiting the scope of the invention.

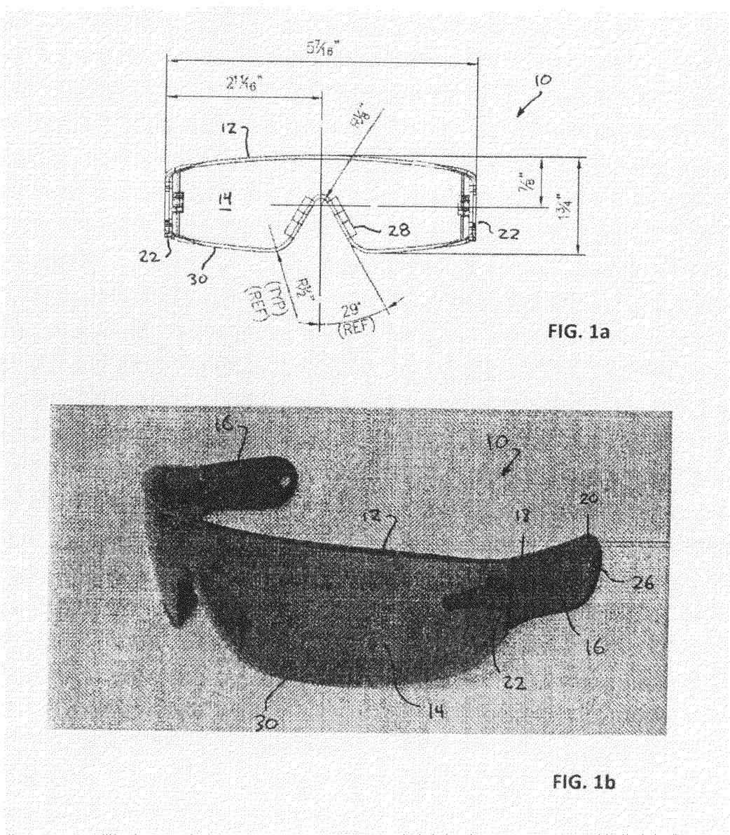

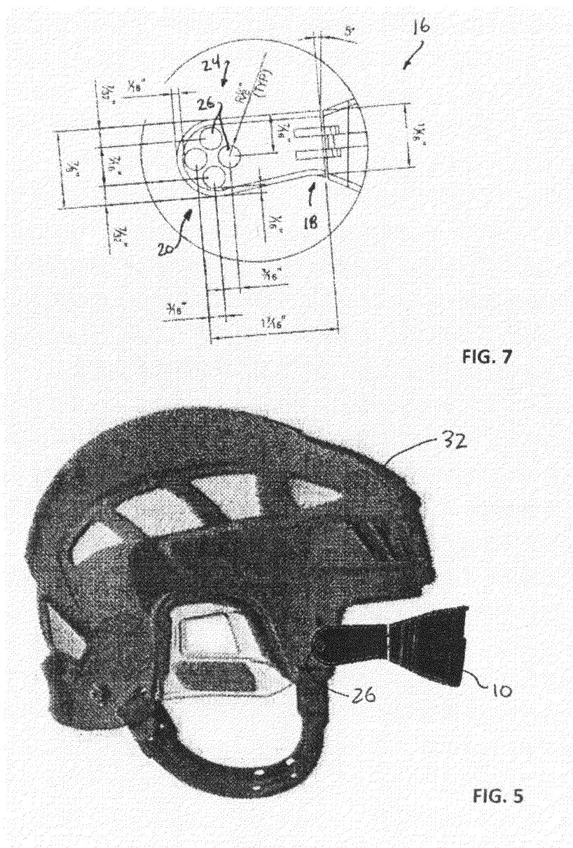

Referring now to FIGS. 1a, 1b, 2 and 3, the eye protection device 10 comprises a frame 12, a transparent viewing lens 14 held within the frame 12, and attachment arms 16. The attachment arms 16 comprise opposed anterior and posterior ends 18, 20, the anterior end 18 configured to mate with a connection location 22 on the frame 12 and the posterior end 20 comprising the attachment means 24 for attaching the device 10 to a sports helmet 32 (the helmet 32 being illustrated in FIG. 5).

The exemplary frame 12 shown in the Figures has a common sunglasses shape, which can simplify manufacture of the device 10. As can be seen in FIG. 1a, the frame does not divide the lens 14, but rather the lens 14 extends across the front length of the device 10, with the frame comprising a nose bridge support 28. The lens 14 is composed of a transparent material with sufficient impact resistance to address the needs of a particular sport, such as a polycarbonate material or other suitable alternative material that would be obvious to one skilled in the art, and in the exemplary embodiment it is seamless for improved visibility and increased strength and impact protection. The lens 14 is also preferably provided with anti-scratch and anti-fog coatings to enhance useful life of the device 10. While the lens 14 is transparent to allow the user to have adequate vision of the surroundings, the frame 12 may be either opaque or transparent. The bottom edges 30 of the frame 12 are rounded for comfort and to avoid injuries from the frame 12 being driven into the user's cheek in an instance of impact.

Referring in detail to FIGS. 1b, 2 and 3, the attachment arms 16 are composed of a transparent, impact-resistant material like polycarbonate, as is the case with the lens 14 itself. Although the device 10 superficially resembles a pair of safety glasses or similar product, it can be seen from the Figures that the attachment arms 16 are not comparable in function or design to standard temple arms on a pair of glasses. The attachment arms 16 are of consistently thick vertical width along the entire length of each arm 16 from the anterior end 18 to the posterior end 20 and terminate well shy of the user's ear, unlike standard safety glasses which taper toward the ear and wrap over or around the ear to hold the glasses in place. This thickened form also provides additional protection to the side of the head not available with most safety glasses. As the length of the arms 16 is only sufficient to enable attachment to helmet 32, this avoids the discomfort encountered when attempting to fit safety glasses arms under a helmet. The actual length of the arms 16 will vary depending on the user, but it is shown in the exemplary embodiment as being 1 7/16inch, much shorter than the common lengths of temple arms on safety glasses. The arms 16 are also preferably provided with rounded posterior ends 20, for comfort in removing the device 10 from a helmet 32.

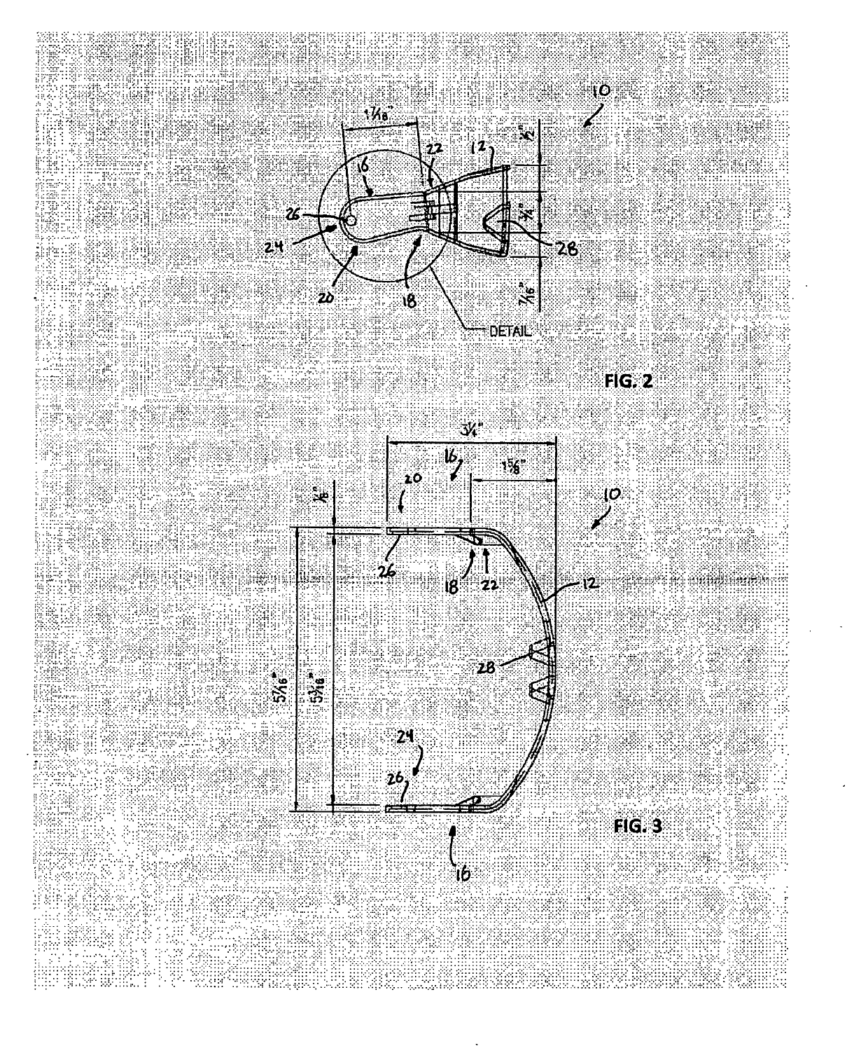

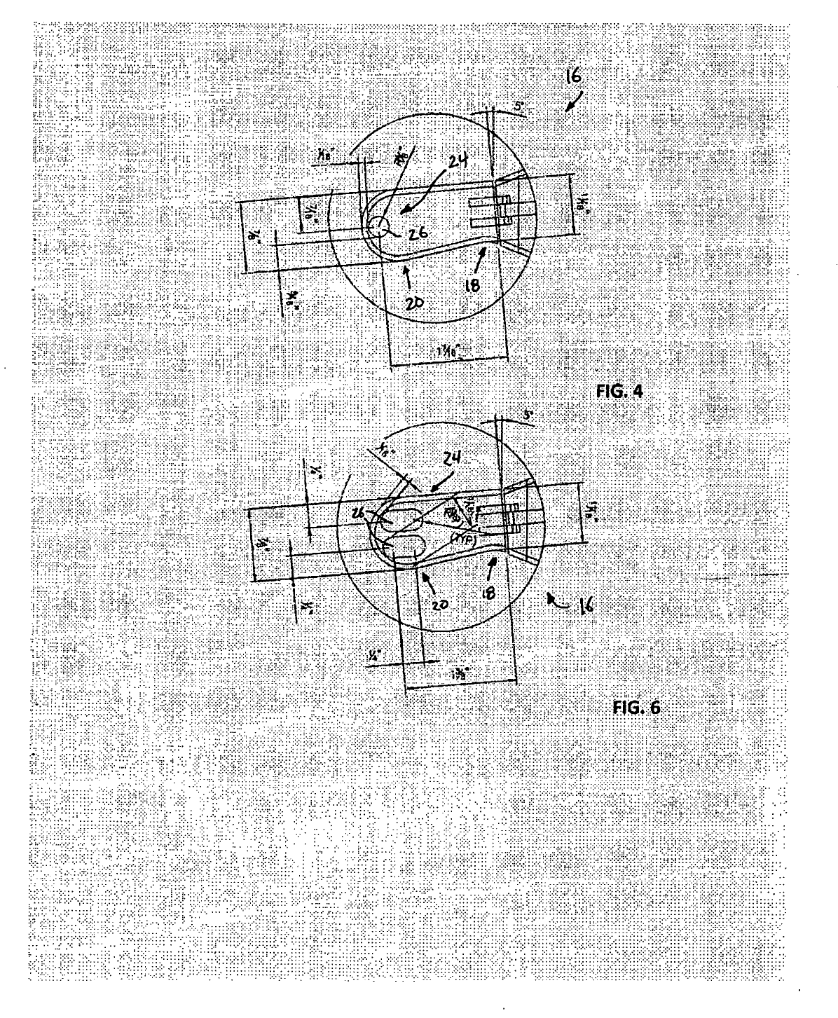

The arms 16 are provided with the attachment means 24 that allow removable attachment of the device 10 to a helmet 32. A detail circle is shown in FIG. 2, and FIGS. 4, 6 and 7 illustrate alternative forms of the attachment means 24 in detail. The preferred attachment means 24 is a simple ¼ inch hole 26 drilled or otherwise formed in the posterior end 20 of each attachment arm 16, as is shown in FIG. 4. This preferred embodiment would be desirable for hockey helmets, as the latter commonly incorporate a bolt at a location adjacent the user's temple which could be used to attach the device 10. The hole 26 would receive the bolt and thereby secure the device 10 in position, in a manner that would be familiar to users and easy to attach and remove. For other styles of sports helmets or where flexibility in sizing is required, alternative attachment means 24 might be desirable. For example, it might be preferably to hove elongate slots 26 as the attachment means 24, as is shown in FIG. 6. Alternatively, a hole 26 might still be the more functional form but different positions might be desired, such that a multiple-hole embodiment as illustrated in FIG. 7 would be the preferred option. These and many other options, including the use of clips and other well-known connection means, would be obvious variants and are intended to be included within the scope of the present invention.

The device 10 would be attached to the helmet 32 by means of these attachment means 24. As can be seen in FIG. 5, the device 10 would be moved into position in front of the helmet 32 such that the holes 26 align with the bolt or other attachment mechanism commonly provided on the helmet 32, and then secured in place during use. The user may choose to disconnect the device 10 at any time with a simple loosening of the bolt from the hole 26.

As can be readily seen, then, the device 10 of the present invention presents significant advantages over the prior art. In being positioned closer to a user's face than commonly-employed visors, the distance between the protective device and the user's face is substantially reduced, hence reducing the risk of injury. Also, a device in accordance with the present invention is less bulky and can weigh significantly less than a standard visor, increasing comfort for the user.

The foregoing is considered as illustrative only of the principles of the invention. For example, the attachment means could comprise a clip on each of the attachment arms to connect to a sports helmet, in a manner that would be obvious to one skilled in the art having access to the within teaching. Thus, while certain aspects and embodiments of the invention have been described, these have been presented by way of example only and are not intended to limit the scope of the invention. Indeed, the invention described herein may be embodied in a variety of other forms without departing from the spirit of the invention, which invention is defined solely by the claims below.

1. An eye protection device for attachment to sports headgear, the device comprising:
a frame;
a viewing lens mounted on the frame;
paired attachment arms each having an anterior end and a posterior end, each of the attachment arms connected at their respective anterior ends to the frame at spaced apart connection locations on the frame; and
each of the attachment arms having attachment means at their respective posterior ends configured for attachment to the sports headgear.

2. The device of claim 1 wherein the attachment means are configured for removable attachment to the sports headgear.

3. The device of claim 1 wherein the viewing lens is held within the frame.

4. The device of claim 1 wherein the respective connection locations are positioned adjacent opposed lateral ends of the frame.

5. The device of claim 1 wherein the attachment arms are of sufficient length to reach opposed lateral sides of the sports headgear for attachment to the sports headgear.

6. The device of claim 1 wherein the attachment means comprise an aperture for receiving a fastening mechanism.

7. The device of claim 6 wherein the aperture comprises one or more holes or slots.

8. The device of claim 6 wherein the fastening mechanism comprises a bolt or clip.

9. The device of claim 1 wherein the attachment arms have a generally consistent vertical thickness from the anterior end to the posterior end.

10. The device of claim 1 wherein the viewing lens is composed of an impact-resistant material.

11. The device of claim 10 wherein the impact-resistant material is polycarbonate.

12. The device of claim 1 wherein the viewing lens is provided with either or both of an anti-scratch coating and an anti-fog coating.

13. The device of claim 1 wherein the frame comprises a nose bridge support.

14. The device of claim 1 wherein the frame comprises a rounded bottom edge.

15. The device of claim 1 wherein the viewing lens extends uninterrupted across a horizontal front extent of the device.

16. The device of claim 1 wherein the viewing lens is of seamless construction.

17. The device of claim 1 wherein the frame is transparent.

18. The device of a claim 1 wherein the frame is opaque.

19. The device of claim 1 wherein the attachment arms are transparent.

20. The device of claim 1 wherein the attachment arms are composed of an impact-resistant material.

21. The device of claim 20 wherein the impact-resistant material is polycarbonate.

22. The device of claim 1 wherein the posterior ends of the attachment arms are rounded.

23. A sports headgear comprising an eye protection device, the eye protection device comprising:
a frame;
a viewing lens mounted on the frame;
paired attachment arms each having an anterior end and a posterior end, each of the attachment arms connected at their respective anterior ends to the frame at spaced apart connection locations on the frame; and
each of the attachment arms having attachment means at their respective posterior ends configured for attachment to attachment locations on the sports headgear.

24. The sports headgear of claim 23 wherein the attachment means are configured for removable attachment to the attachment locations.

25. The sports headgear of claim 23 wherein the viewing lens is held within the frame.

26. The sports headgear of claim 23 wherein the respective connection locations are positioned adjacent opposed lateral ends of the frame.

27. The sports headgear of claim 23 wherein the attachment arms are of sufficient length to reach the attachment locations.

28. The sports headgear of claim 23 wherein the attachment means comprise an aperture for receiving a fastening mechanism.

29. The sports headgear of claim 28 wherein the aperture comprises one or more holes or slots.

30. The sports headgear of claim 28 wherein the fastening mechanism comprises a bolt or clip.

31. The sports headgear of claim 23 wherein the attachment arms have a generally consistent vertical thickness from the anterior end to the posterior end.

32. The sports headgear of claim 23 wherein the viewing lens is composed of an impact-resistant material.

33. The sports headgear of claim 33 wherein the impact-resistant material is polycarbonate.

34. The sports headgear of claim 23 wherein the viewing lens is provided with either or both of an anti-scratch coating and an anti-fog coating.

35. The sports headgear of claim 23 wherein the frame comprises a nose bridge support.

36. The sports headgear of claim 23 wherein the frame comprises a rounded bottom edge.

37. The sports headgear of claim 23 wherein the viewing lens extends uninterrupted across a horizontal front extent of the device.

38. The sports headgear of claim 23 wherein the viewing lens is of seamless construction.

39. The sports headgear of claim 23 wherein the frame is transparent.

40. The sports headgear of claim 23 wherein the frame is opaque.

41. The sports headgear of claim 23 wherein the attachment arms are transparent.

42. The sports headgear of claim 23 wherein the attachment arms are composed of an impact-resistant material.

43. The sports headgear of claim 42 wherein the impact-resistant material is polycarbonate.

44. The sports headgear of claim 23 wherein the posterior ends of the attachment arms are rounded.
